(19) United States
(12) Patent Application Publication
(10) Pub. No.: US 2025/0372264 A1
(43) Pub. Date: Dec. 4, 2025

(54) IMAGE PROCESSING DEVICE, IMAGE PROCESSING METHOD, AND STORAGE MEDIUM

(71) Applicant: NEC Corporation, Minato-ku, Tokyo (JP)

(72) Inventors: Kazuhiro WATANABE, Tokyo (JP); Ryosaku SHINO, Tokyo (JP); Akinori EBIHARA, Tokyo (JP); Taiki MIYAGAWA, Tokyo (JP)

(73) Assignee: NEC Corporation, Minato-ku, Tokyo (JP)

(21) Appl. No.: 18/877,742

(22) PCT Filed: Jul. 12, 2022
(86) PCT No.: PCT/JP2022/027406

§ 371 Date: Dec. 20, 2024

(51) Int. Cl. G16H 50/30; G06T 7/00; G16H 30/20

(52) U.S. Cl. CPC G16H 50/30; G06T 7/0012; G16H 30/20; G06T 2207/10068; G06T 2207/30096

(57) ABSTRACT

The image processing device 1X includes an acquisition means 30X, a score calculation means 31X, and a classification means 32X. The acquisition means 30X is configured to acquire an endoscopic image in which an examination target is photographed by a photographing unit provided in an endoscope. The score calculation means 31X is configured to calculate scores on likelihoods of respective candidate classes corresponding to types of lesion, wherein the candidate classes are candidates for classification of an image group of the acquired endoscopic image. The classification means 32X is configured to perform the classification of the image group upon determining that at least one of the scores has reached a threshold value.

### TECHNICAL FIELD

The present disclosure relates to a technical field of an image processing device, an image processing method, and a storage medium for processing an image to be acquired in endoscopic examination.

### BACKGROUND

An endoscopic examination system for displaying images taken in the lumen of an organ is known. For example, Patent Literature 1 discloses a learning method of a learning model that outputs information relating to a lesion part included in an endoscope image data upon receiving the endoscope image data generated by a photographing device. Further, Patent Literature 2 discloses a classification method for classifying series data through an application method of the sequential probability ratio test (SPRT: Sequential Probability Ratio Test). Further, Non-Patent Literature 1 discloses an approximate computation method of the matrix for multi-class classification in the SPRT-based technique according to Patent Literature 2.

### CITATION LIST

### Patent Literature

Patent Literature 1: WO2020/003607
Patent Literature 1: WO2020/194497

### Non-Patent Literature

Non-Patent Literature 1: Miyagawa Taiki, and Akinori F. Ebihara. “The Power of Log-Sum-Exp: Sequential Density Ratio Matrix Estimation for Speed-Accuracy Optimization.” International Conference on Machine Learning. PMLR, 2021.

### SUMMARY

### Problem to be Solved

In the case of classifying a lesion (i.e., making a qualitative diagnosis) based on an image photographed in the endoscopic examination, there is a possibility that a lesion part which is difficult to be identified from a single image could not be classified properly. In contrast, in the case of classifying a lesion part from a plurality of images, there is an issue that it is difficult to set the appropriate number of images to be used for the classification.

In view of the above-described issue, it is therefore an example object of the present disclosure to provide an image processing device, an image processing method, and a storage medium capable of suitably classifying a lesion part in an endoscopic image.

### Means for Solving the Problem

One mode of the image processing device is an image processing device including:
an acquisition means configured to acquire an endoscopic image in which an examination target is photographed by a photographing unit provided in an endoscope;
a score calculation means configured to calculate scores on likelihoods of respective candidate classes corresponding to types of lesion, wherein the candidate classes are candidates for classification of an image group of the acquired endoscopic image; and a classification means configured to perform the classification of the image group upon determining that at least one of the scores has reached a threshold value.

One mode of the image processing method is an image processing method executed by a computer, the image processing method including:
acquiring an endoscopic image in which an examination target is photographed by a photographing unit provided in an endoscope;
calculating scores on likelihoods of respective candidate classes corresponding to types of lesion, wherein the candidate classes are candidates for classification of an image group of the acquired endoscopic image; and
performing the classification of the image group upon determining that at least one of the scores has reached a threshold value.

One mode of the storage medium is a storage medium storing a program executed by a computer, the program causing the computer to:
acquire an endoscopic image in which an examination target is photographed by a photographing unit provided in an endoscope;
calculate scores on likelihoods of respective candidate classes corresponding to types of lesion, wherein the candidate classes are candidates for classification of an image group of the acquired endoscopic image; and
perform the classification of the image group upon determining that at least one of the scores has reached a threshold value.

### Effect

An example advantage according to the present disclosure is to suitably classify a lesion part in an endoscopic image.

### BRIEF DESCRIPTION OF THE DRAWINGS

FIG. 1 It illustrates a schematic configuration of an endoscopic examination system.

FIG. 2 It illustrates a hardware configuration of an image processing device.

FIG. 3 It is a functional block diagram of the image processing device.

FIG. 4 It illustrates a graph indicating the transition of classification scores.

FIG. 5 It illustrates a first display example of the display screen image displayed on a display device in the endoscopic examination.

FIG. 6 It illustrates a second display example of the display screen image displayed on the display device in the endoscopic examination.

FIG. 7 It illustrates a third display example of the display screen image displayed on the display device in the endoscopic examination.

FIG. 8 It is an example of a flowchart executed by the image processing device.

FIG. 9 It is a block diagram of an image processing device according to the second example embodiment.

FIG. 10 An example of a flowchart executed by the image processing device in the second example embodiment.

### EXAMPLE EMBODIMENTS

Hereinafter, example embodiments of an image processing device, an image processing method, and a storage medium will be described with reference to the drawings.

### First Example Embodiment

### (1) System Configuration

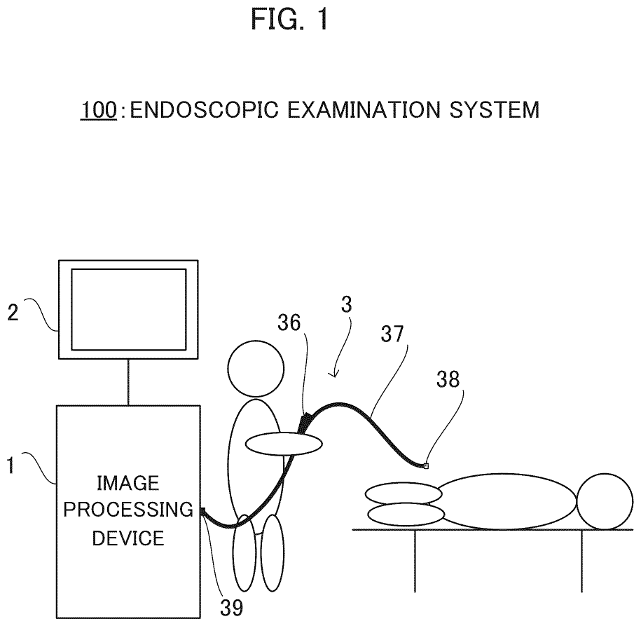

FIG. 1 illustrates a schematic configuration of an endoscopic examination system 100. The endoscopic examination system 100 makes a qualitative diagnosis to classify a part (lesion part) suspected of a lesion in an examination target and provides the classification result to an examiner such as a doctor who conducts examination or treatment using an endoscope. The endoscopic examination system 100, as shown in FIG. 1, mainly includes an image processing device 1, a display device 2, and an endoscope 3 connected to the image processing device 1.

The image processing device 1 acquires an image (also referred to as “endoscopic image Ia”) captured by the endoscope 3 in time series from the endoscope 3 and displays a screen image based on the endoscopic image Ia on the display device 2. The endoscopic image Ia is an image captured at predetermined time intervals in at least one of the insertion process of the endoscope 3 to the subject or the ejection process of the endoscope 3 from the subject. In the present example embodiment, upon detecting an endoscopic image Ia (referred to as “lesion image”) in which a lesion part is shown, the image processing device 1 classifies the lesion part based on time-series lesion images and causes the display device 2 to display information regarding the classification result.

The display device 2 is a display or the like for display information based on the display signal supplied from the image processing device 1.

The endoscope 3 mainly includes an operation unit 36 for examiner to perform a predetermined input, a shaft 37 which has flexibility and which is inserted into the organ to be photographed of the subject, a tip unit 38 having a built-in photographing unit such as an ultra-small image pickup device, and a connecting unit 39 for connecting with the image processing device 1. In the present exemplary embodiment, the operation unit 36 includes a button (also referred to as “still image saving button”) to capture (i.e., save as a still image) an endoscopic image displayed on the display device 2 when the examiner determines that an endoscopic image showing a tumor part is displayed on the display device 2.

The configuration of the endoscopic examination system 100 shown in FIG. 1 is an example, and various change may be applied thereto. For example, the image processing device 1 may be configured integrally with the display device 2. In another example, the image processing device 1 may be configured by a plurality of devices.

Hereafter, as a representative example, the description will be given of the process in the endoscopic examination of the large bowel. However, the examination target is not limited to the large bowel and it may be an esophagus or stomach. Examples of the target of the endoscopic examination in the present disclosure include a laryngendoscope, a bronchoscope, an upper digestive tube endoscope, a duodenum endoscope, a small bowel endoscope, a large bowel endoscope, a capsule endoscope, a thoracoscope, a laparoscope, a cystoscope, a cholangioscope, an arthroscope, a spinal endoscope, a blood vessel endoscope, and an epidural endoscope. In addition, the conditions of the lesion part to be detected in endoscopic examination are exemplified as (a) to (f) below.
(a) Head and neck: pharyngeal cancer, malignant lymphoma, papilloma
(b) Esophagus: esophageal cancer, esophagitis, esophageal hiatal hernia, Barrett's esophagus, esophageal varices, esophageal achalasia, esophageal submucosal tumor, esophageal benign tumor
(c) Stomach: gastric cancer, gastritis, gastric ulcer, gastric polyp, gastric tumor
(d) Duodenum: duodenal cancer, duodenal ulcer, duodenitis, duodenal tumor, duodenal lymphoma
(e) Small bowel: small bowel cancer, small bowel neoplastic disease, small bowel inflammatory disease, small bowel vascular disease
(f) Large bowel: colorectal cancer, colorectal neoplastic disease, colorectal inflammatory disease; colorectal polyps, colorectal polyposis, Crohn's disease, colitis, intestinal tuberculosis, hemorrhoids.

### (2) Hardware Configuration

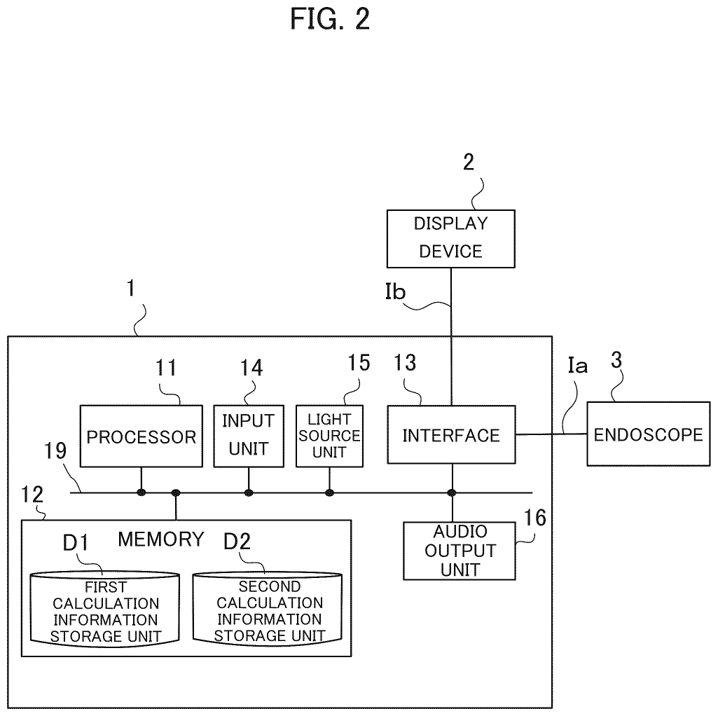

FIG. 2 shows the hardware configuration of the image processing device 1. The image processing device 1 mainly includes a processor 11, a memory 12, an interface 13, an input unit 14, a light source unit 15, and an audio output unit 16. Each of these elements is connected via a data bus 19.

The processor 11 executes a predetermined process by executing a program or the like stored in the memory 12. The processor 11 is one or more processors such as a CPU (Central Processing Unit), a GPU (Graphics Processing Unit), and a TPU (Tensor Processing Unit). The processor 11 may be configured by a plurality of processors. The processor 11 is an example of a computer.

The memory 12 is configured by a variety of volatile memories which are used as working memories, and nonvolatile memories which store information necessary for the process to be executed by the image processing device 1, such as a RAM (Random Access Memory) and a ROM (Read Only Memory). The memory 12 may include an external storage device such as a hard disk connected to or built in to the image processing device 1, or may include a storage medium such as a removable flash memory. The memory 12 stores a program for the image processing device 1 to execute each process in the present example embodiment.

The memory 12 functionally includes a first calculation information storage unit D1 for storing first calculation information and a second calculation information storage unit D2 for storing second calculation information. The first calculation information and the second calculation information are information used by the image processing device 1 in the classification of a lesion part or information indicating the results of the calculation relating to the classification, and the details thereof will be described later. In addition, the memory 12 stores various parameters necessary for calculating the score of the classification of a lesion part. At least a portion of the information stored in the memory 12 may be stored in an external device other than the image processing device 1. In this case, the above-described external device may be one or more server devices capable of data communication with the image processing device 1 through a communication network or through direct communication.

The interface 13 performs an interface operation between the image processing device 1 and an external device. For example, the interface 13 supplies the display information “Ib” generated by the processor 11 to the display device 2. Further, the interface 13 supplies the light generated by the light source unit 15 to the endoscope 3. The interface 13 also provides an electrical signal to the processor 11 indicative of the endoscopic image Ia supplied from the endoscope 3. The interface 13 may be a communication interface, such as a network adapter, for wired or wireless communication with the external device, or a hardware interface compliant with a USB (Universal Serial Bus), a SATA (Serial AT Attachment), or the like.

The input unit 14 generates an input signal based on the operation by the examiner. Examples of the input unit 14 include a button, a touch panel, a remote controller, and a voice input device. The light source unit 15 generates light for supplying to the tip unit 38 of the endoscope 3. The light source unit 15 may also incorporate a pump or the like for delivering water and air to be supplied to the endoscope 3. The audio output unit 16 outputs a sound under the control of the processor 11.

### (3) Outline of Lesion Part Detection Process

Next, an outline of the process of detecting a lesion part by the image processing device 1 will be described. In summary, the image processing device 1 classifies the lesion part based on a variable number of time series endoscopic images Ia. Thus, the image processing device 1 accurately classifies the lesion part which is difficult to classify from a single image, and presents the classification result.

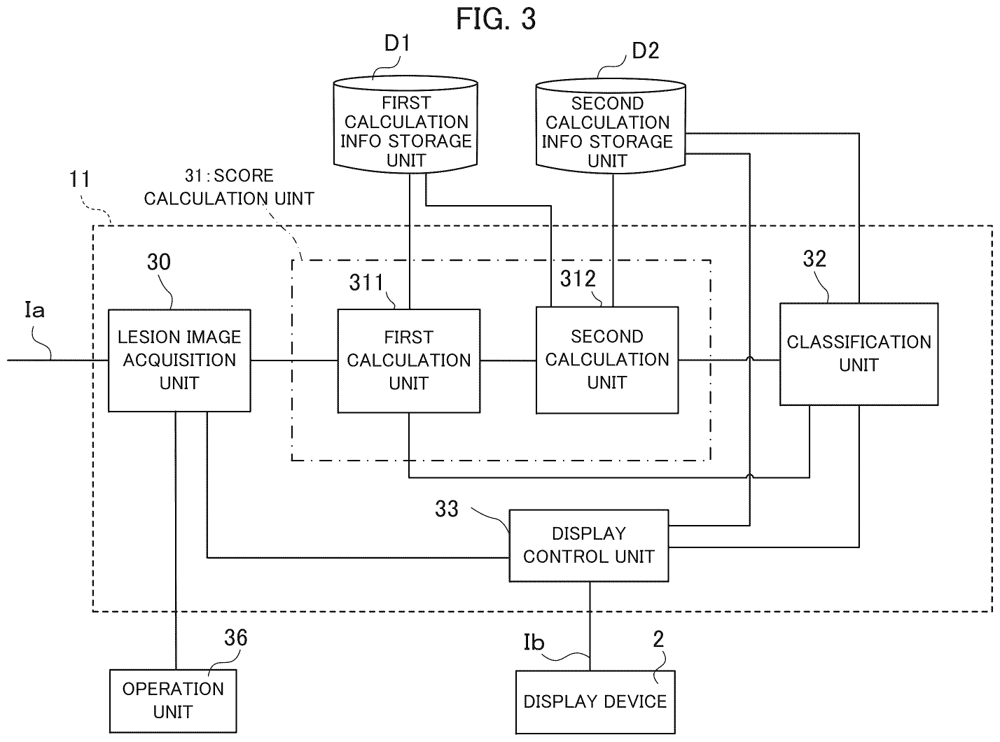

FIG. 3 is a functional block diagram of the image processing device 1. As shown in FIG. 3, the processor 11 of the image processing device 1 functionally includes a lesion image acquisition unit 30, a score calculation unit 31, a classification unit 32, and a display control unit 33. In FIG. 3, blocks to transmit and receive data to or from each other are connected by a solid line, but the combination of blocks to transmit and receive data to or from each other is not limited to FIG. 3. The same applies to the drawings of other functional blocks described below.

The lesion image acquisition unit 30 acquires endoscopic images Ia taken by the endoscope 3 through the interface 13 at predetermined intervals according to the frame period of the endoscope 3, and selects one or more lesion images from the acquired endoscopic images Ia. Then, the lesion image acquisition unit 30 supplies the selected lesion images to the score calculation unit 31 and the display control unit 33. Further, the lesion image acquisition unit 30 supplies the acquired endoscopic images Ia to the display control unit 33.

Here, a specific example of a method for selecting a lesion image will be described. For example, upon detecting selection of the still image saving button based on a signal supplied from the operation unit 36 (i.e., an external input based on user operation), the lesion image acquisition unit 30 acquires the endoscopic image Ia displayed on the display device 2 at the time of the selection as a lesion image. In this instance, the lesion image acquisition unit 30 may acquire, as the lesion image, the most recent endoscopic image Ia received from the endoscope 3 at the time when the still image saving button has been selected.

In another example, the lesion image acquisition unit 30 may select the lesion image without depending on the operation (i.e., external input) by the examiner. For example, on the basis of a model (also referred to as “lesion detection model”) configured to detect a lesion image, the lesion image acquisition unit 30 may acquire the lesion image. In this case, the parameters of the lesion detection model are stored in the memory 12 or the like in advance. The lesion image acquisition unit 30 builds the lesion detection model by referring to the above-described parameters and inputs an endoscope image Ia supplied from the endoscope 3 to the lesion detection model. Then, on the basis of the information outputted by the lesion detection model in response to the inputted endoscopic image Ia, the lesion image acquisition unit 30 determines whether or not the inputted endoscopic image Ia is a lesion image. In this case, the lesion detection model is, for example, a classification model that is trained to output a classification result regarding the presence or absence of a lesion part in the endoscopic image Ia upon receiving an endoscopic image Ia. For example, when the lesion detection model is configured based on a neural network, various parameters such as a layer structure, a neuron structure of each layer, the number of filters and the size of filters in each layer, and the weight for each element of each filter are previously stored in the memory 12 or the like.

In some embodiments, the display control unit 33, which will be described later, may display the lesion detection result based on the lesion detection model together with the most recent endoscopic image Ia to thereby support the operation of the still image saving button by the examiner. For example, upon detecting the lesion part by the lesion detection model, the display control unit 33 may prompt the examiner to press the still image saving button by highlighting the most recent endoscopic image Ia displayed on the display device 2 by the edging effect or the like.

Then, at the time intervals of acquisition of the lesion image by the lesion image acquisition unit 30, the score calculation unit 31, the classification unit 32, and the display control unit 33 perform the processing described later in a cycle. Hereafter, the timing of the processing based on the cycle is also referred to as “processing time”.

With respect to each of candidates (referred to as “candidate classes”) for a class to which the lesion part belongs, the score calculation unit 31 calculate a score (also referred to as “classification score”) indicating the likelihood that the lesion part belongs to the candidate class, wherein the classification score is used for classification regarding the class into which the lesion part should be classified. In this case, the score calculation unit 31 calculates the classification scores for respective candidate classes using the time series lesion images according to a SPRT based method described in Patent Literature 2 and Non-Patent Literature 1. The number of candidate classes and the types of the lesion part corresponding to the respective candidate classes are set in advance for each examination target.

The score calculation unit 31 functionally includes a first calculation unit 311 and a second calculation unit 312.

The first calculation unit 311 calculates, for each processing time, a likelihood ratio regarding the latest “N” (N is an integer) lesion images, and supplies the calculation result to the second calculation unit 312. The “likelihood ratio” is an index indicating the likelihood that the lesion images belong to a class, and the likelihood ratio increases when the correct answer class is in the numerator of the likelihood ratio, and decreases when the correct answer class is in the denominator of the likelihood ratio. In this case, the first calculation unit 311 calculates the likelihood ratio using the likelihood ratio calculation model that has been trained to output the likelihood ratio for the inputted N lesion images when N lesion images are inputted to the likelihood ratio calculation model, for example. The likelihood ratio calculation model may be a deep learning model, or any other machine learning model or a statistical model. In this case, for example, learned parameters of the likelihood ratio calculation model is stored in the memory 12, and the first calculation unit 311 inputs the latest N lesion images to the likelihood ratio calculation model configured by referring to the parameters and acquires the likelihood ratio outputted by the model. If the likelihood ratio calculation model is constituted by a neural network, various parameters such as a layer structure, a neuron structure of each layer, the number of filters and filter sizes in each layer, and a weight for each element of each filter are previously stored in the memory 12. Even if only less than N lesion images are acquired, the first calculation unit 311 can acquire the likelihood ratio using the likelihood ratio calculation model and the less than N lesion images.

The likelihood ratio calculation model may include an arbitrary feature extractor for extracting features (i.e., feature vector) of each lesion image that is inputted into the likelihood ratio calculation model, or may be configured separately from the feature extractor. In the latter case, the likelihood ratio calculation model is a model trained to output likelihood ratios of respective candidate classes regarding the N lesion images upon receiving features of N lesion images extracted by the feature extractor. In some embodiments, the feature extractor may extract the features representing the relation among time series data based on any technique for calculating the relation among time series data such as LSTM (Long Short Term Memory).

In some embodiments, the first calculation unit 311 sets the number N in accordance with the type of the examination target. For example, in such a case that the examination target is an organ (for example, the stomach) in which the endoscope can be moved to a certain extent, the first calculation unit 311 sets the number N to a value smaller than the value in the case of the other types of the examination target since the correlation between the lesion images becomes relatively small. On the other hand, in such a case that the examination target is an organ (e.g., esophagus) in which the endoscope cannot be almost moved, the first calculation unit 311 sets the number N to a value larger than the value in the case of the other types of the examination target since the correlation between the lesion images becomes relatively large. Thus, the first calculation unit 311 can calculate the likelihood ratio more accurately. Similarly, likelihood ratio calculation models may be prepared for respective types of the examination target. In this case, the likelihood ratio calculation models are trained for respective types of the examination target, and parameters obtained through the training are stored in advance in the memory 12 or the like for each type of the examination target. The image processing device 1 may recognize the type of the examination target based on an external input or the like by the input unit 14 prior to the endoscopic examination, or may automatically recognize the examination target by applying any image recognition technique to the endoscopic image Ia obtained at the beginning of the endoscopic examination.

As the first calculation information, the first calculation unit 311 stores, in the first calculation information storage unit D1, the calculated likelihood ratio and data used for calculation of the likelihood ratio by the first calculation unit 311. The “data used for calculation of the likelihood ratio” may be lesion images used for calculation of the likelihood ratio, or may be features extracted from the lesion images.

The second calculation unit 312 calculates a likelihood ratio (also referred to as “integrated likelihood ratio”) obtained by integrating the likelihood ratios calculated in time series, and determines the classification score based on the integrated likelihood ratio. The classification score may be the integrated likelihood ratio itself or may be a function including the integrated likelihood ratio as a variable.

Here, for simplicity of explanation, first, a specific method of calculating the integrated likelihood ratio in the case of performing binary classification will be described.

The time index “t” indicates the current processing time on the assumption that the time index “1” indicates the time when a lesion image was firstly obtained, and any target lesion image or its features of processing is set to “xᵢ” (i=1, . . . , t). Here, the time index shall increase one-by-one every time a lesion image is obtained. It is noted that t lesion images subject to processing is an example of “image group”.

Here, on the assumption that the candidate class “C₀” and the candidate class “C₁” are provided, the integrated likelihood ratio of the candidate class C₁ is expressed by the following equation (1).

$$log [ \frac{p ( {x}_{1} , … , {x}_{t} \vert {C}_{1} )}{p ( {x}_{1} , … , {x}_{t} \vert {C}_{0} )} ] = {\sum }_{s = N + 1}^{t} log [ \frac{p ( {C}_{1} \vert {x}_{s} , … , {x}_{s - N} )}{p ( {C}_{0} \vert {x}_{s} , … , {x}_{s - N} )} ] - {\sum }_{s = N + 2}^{t} log [ \frac{p ( {C}_{1} \vert {x}_{s - 1} , … , {x}_{s - N} )}{p ( {C}_{0} \vert {x}_{s - 1} , … , {x}_{s - N} )} ] \qquad [ Equation 1 ]$$

Here, “p” represents the probability of each candidate class (i.e., the confidence level with the range of 0 to 1). In calculating the term on the right side of the equation (1), it is possible to use the likelihood ratio stored in the first calculation information storage unit D1 as the first calculation information by the first calculation unit 311. The integrated likelihood ratio for the candidate class C₀ is the inverse of the equation (1).

Regarding the equation (1), since the time index t which represents the current process time increases with the elapse of time, the length of the time series lesion images (or the features thereof) used for calculating the integrated likelihood ratio is a variable length. Thus, by using the integrated likelihood ratio based on the equation (1), the second calculation unit 312 can compute the classification score while considering a variable number of lesion images as a first advantage. In addition, by using the integrated likelihood ratio based on the equation (1), it is possible to classify the time-dependent features as the second advantage. As the third advantage, the classification score allowing for a robust classification can be suitably calculated even when difficult-to-identify data is used.

Next, a description will be given of the calculation of the integrated likelihood ratio of each candidate class in the case where classification (multi-class classification) of three or more classes is performed. Assuming that the number of candidate classes is “M” (M is an integer of 3 or more), the score calculation unit 31 calculates the integrated likelihood ratio between the k-th (k=1, 2, . . . , M) candidate class and all remaining classes among the M candidate classes. In this case, for example, the score calculation unit 31 calculates the integrated likelihood ratio using the equation (1) while replacing the denominators of the first term and the second term on the right side of the equation (1) with the maximum likelihood among all candidate classes other than the k-th candidate class. In this case, the score calculation unit 31 may calculate the integrated likelihood ratio using the sum of the likelihoods of all candidate classes other than the k-th candidate class, instead of using the maximum likelihood. Therefore, for example, the score calculation unit 31 calculates the integrated likelihood ratio of each candidate class based on the likelihood ratio (i.e., the likelihood ratio shown on the right side of the equation (1)) of each candidate class outputted by the likelihood ratio calculation model upon inputting the target N lesion images of feature extraction by the feature extractor or the features thereof to the likelihood ratio calculation model. It is noted that examples of the calculation method of the integrated likelihood ratio and the classification score include not only the above-described methods but also the methods described in Patent Literature 2 and Non-Patent Literature 1.

The second calculation unit 312 stores in the second calculation information storage unit D2 the integrated likelihood ratios and the classification scores of each candidate class calculated at process times at which the lesion images are obtained, as the second calculation information.

Based on the classification score calculated by the second calculation unit 312, the classification unit 32 performs a classification regarding the lesion part, and supplies the classification result to the display control unit 33. In this instance, the classification unit 32 compares the classification score of the lesion part for each candidate class with a predetermined threshold value (also referred to as “threshold value Th”), and determines whether or not there is a candidate class having the classification score equal to or larger than the threshold value Th.

Then, if there is a candidate class having the classification score equal to or larger than the threshold value Th, the classification unit 32 outputs the candidate class having the classification score equal to or larger than the threshold value Th as the classification result of the lesion part appearing in the lesion image group used for calculation of the classification score. It is herein assumed that the classification score of a candidate class increases with increasing probability that the lesion part, which appears in the lesion image group used for calculation of the classification score, belongs to the candidate class. For example, the threshold value TH is set to a calibration value determined through experimental trials or the like, and is stored in advance in the memory 12 or the like. Thereafter, the classification unit 32 supplies a notification for resetting the calculation process of the classification score (i.e., updating the start time) to the score calculation unit 31.

On the other hand, upon determining that there is no candidate class whose classification score is equal to or larger than the threshold value TH, the classification unit 32 instructs the score calculation unit 31 to calculate the classification score for the lesion image group to which a lesion image acquired by the lesion image acquisition unit 30 after the determination is added.

Even when it is determined that there is no candidate class whose classification score is equal to or larger than the threshold value TH, the classification unit 32 may determine the classification as long as a predetermined condition which is determined in advance other than the condition based on the threshold value TH is satisfied. For example, if the time index t representing the current processing time becomes equal to or larger than a predetermined threshold value (i.e., if the number of the lesion images in the image group to be used becomes a predetermined number or more), the classification unit 32 may determine the classification. In this case, once the time index t representing the current processing time becomes a predetermined threshold value or more, the classification unit 32 outputs the classification result of the lesion part shown in the lesion images used for calculation of the classification score, wherein the classification result indicates the candidate class having the highest classification score. The set value of the predetermined threshold (predetermined number) described above, for example, is stored in advance in the memory 12 or the like.

In some embodiments, if it is determined that there is no candidate class having the classification score equal to or more than the threshold value TH and that the time index t representing the current processing time becomes equal to or more than the predetermined threshold value (that is, the number of the lesion images in the image group to be used becomes a predetermined number or more), the classification unit 32 may determine that the classification score should be reset. In this case, the classification unit 32 instructs the score calculation unit 31 to reset the calculation process of the classification score (i.e., update the start time), without determining the classification. In this case, the first calculation unit 311 and the second calculation unit 312 of the score calculation unit 31 reset the classification scores (as well as the first calculation information and the second calculation information) of respective candidate classes and calculate the classification scores based on the lesion image group newly acquired from the time when the reset is performed. The set value of the predetermined threshold value (predetermined number) described above, for example, is stored in advance in the memory 12 or the like.

The display control unit 33 generates the display information Ib based on the endoscopic images Ia (including the lesion images), the classification result supplied from the classification unit 32, and supplies the display device 2 with the display information Ib through the interface 13, thereby causing the display device 2 to display information regarding the endoscopic images Ia and the classification result by the classification unit 32. In some embodiments, the display control unit 33 may cause the display device 2 to further display information on the classification score stored in the second calculated information storage unit D2. The display example of the display control unit 33 will be described later.

Each component of the lesion image acquisition unit 30, the score calculation unit 31, the classification unit 32, and the display control unit 33 can be realized, for example, by the processor 11 which executes a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

### (4) Calculation Example of Classification Score

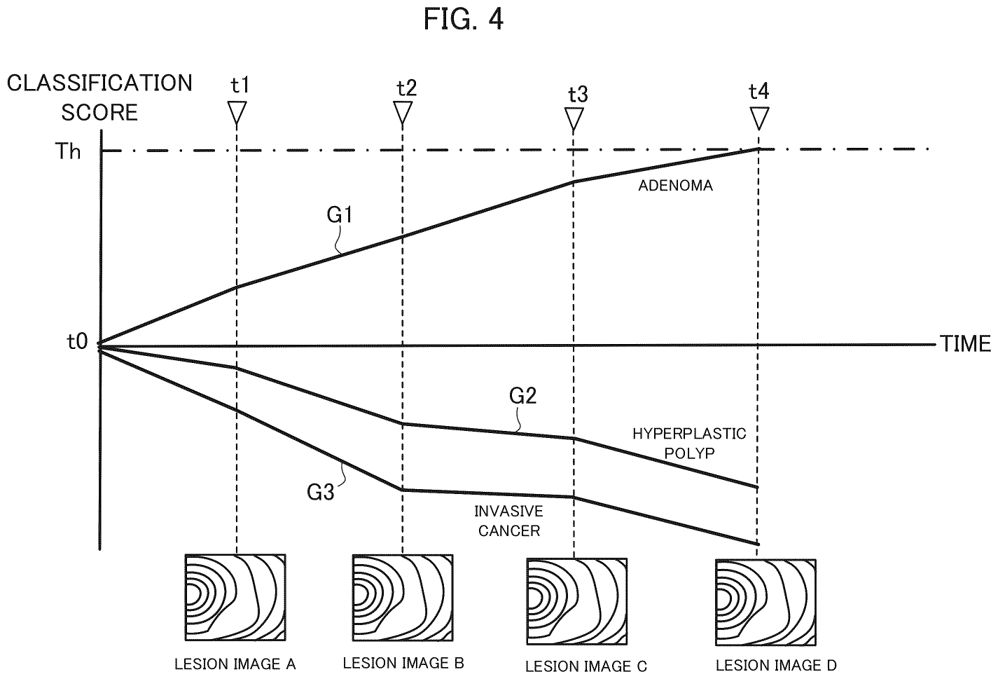

Next, an example of calculating the classification score will be described. FIG. 4 illustrates graphs showing the transition of the classification scores. In this example, the image processing device 1 starts the process at the time “t0” and calculates classification scores of three candidate classes (“adenoma”, “hyperplastic polyp”, and “invasive cancer”) on the basis of the lesion images (the lesion images A to the lesion images D) that the lesion image acquisition unit 30 acquires at the time “t1,” “t2,” “t3,” and “t4,” respectively. Here, the graph G1 shows the transition of the classification score of the candidate class “adenoma”, the graph G2 shows the transition of the classification score of the candidate class “hyperplastic polyp”, and the graph G3 shows the transition of the classification score of the candidate class “invasive cancer”.

First, at the time t1, the image processing device 1 calculates the classification scores of the candidate classes based on the lesion images A obtained at the time t1. At the time t2, the image processing device 1 calculates the classification scores of the candidates based on the lesion image B obtained at the time t2 and the lesion image A obtained at the time t1. Furthermore, the image processing device 1 calculates the classification scores of the respective candidate classes based on the lesion image C obtained at the time t3 and the lesion image A and the lesion image B obtained in the past. At the time t4, the image processing device 1 calculates the classification scores of respective candidate classes based on the lesion image D obtained at the time t4 and the lesion image A to the lesion image C obtained in the past.

Then, as shown in the graph G1, at the time t4, since the classification score of the candidate class “adenoma” is equal to or larger than the threshold value TH, the image processing device 1 generates such a classification result that the lesion part appearing in the lesion image A to the lesion image D obtained from the time to to the time t4 is “adenoma”.

Thus, the image processing device 1 sequentially calculates the classification scores for the image group including one or more input images, and performs classification once a classification score reaches the threshold value. Thus, it is possible to suitably improve the classification performance by using an image group including an optimum number of images for classification without excess or deficiency. On the other hand, if a fixed number of images is used to classify the lesion, there is a problem that it is difficult to fix the number of images to be used so as to be optimum for the classification, because the quality of the individual images to be used is not taken into consideration. For example, when the number of images to be used is large, there is a possibility that images with noise such as blur may be included due to an increase in the number of images to be used, and when the number of images to be used is small, classification will be performed in a condition where the confidence degree of classification is small, and in both cases, the possibility of misclassification increases. Taking the above into consideration, the image processing device 1 according to the example embodiment uses a variable number of images, and performs classification once the classification score reaches a threshold value. It allows for suitable improvement in the classification performance using an optimum number of images for classification without excess or deficiency.

### (5) Display Example

Next, a description will be given of the display control of the display device 2 to be executed by the display control unit 33.

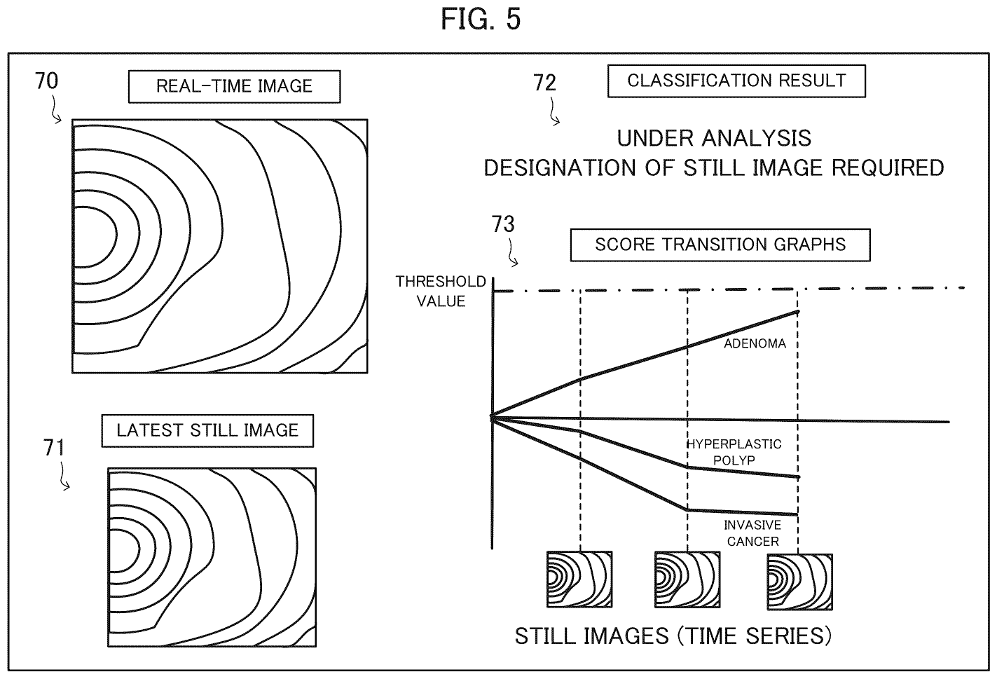

FIG. 5 shows a first display example of a display screen image displayed on the display device 2 in the endoscopic examination. The display control unit 33 of the image processing device 1 outputs to the display device 2 the display information Ib generated based on the endoscopic images Ia and lesion images acquired by the lesion image acquisition unit 30 and the classification result generated by the classification unit 32. The display control unit 33 transmits the endoscopic images Ia and the display information Ib to the display device 2 to thereby cause the display device 2 to display the above-described display screen image. Further, in this example, the lesion image acquisition unit 30 acquires a still image specified as a lesion image based on the operation to the operation unit 36 by the examiner.

In the first display example, on the display screen image, the display control unit 33 of the image processing device 1 provides a real-time image display area 70, a latest still image display area 71, a classification result display area 72, and a score transition display area 73.

Here, the display control unit 33 displays, in the real-time image display area 70, a moving image representing the most recent endoscopic image Ia. Further, the display control unit 33 displays, in the latest still image display area 71, the latest still image (i.e. the most recent lesion image acquired by the lesion image acquisition unit 30).

Furthermore, in the classification result display area 72, the display control unit 33 displays the classification result generated by the classification unit 32. At the display time of the display screen image shown in FIG. 5, since the classification result of any of the candidate classes has not reached the threshold value TH, the display control unit 33 displays, in the classification result display area 72, a text message to the effect that it is under analysis and designation of still images (i.e. lesion images) is required.

Further, in the score transition display area 73, the display control unit 33 displays score transition graphs (in this case, a diagram corresponding to FIG. 4) indicating the transition of the classification score of each candidate class from the start point of the endoscopic examination to the present time. In this case, in the score transition graphs, the display control unit 33 displays still images (i.e., lesion images) used for calculating the classification scores in association with the time when the respective still images were specified. Thus, the display control unit 33 can present the relation between the obtained still image and the variation in the classification score to the examiner. The score transition graphs displayed on the score transition display area 73 are an example of the “diagram showing a transition of the scores”.

Thus, according to the first display example, when the classification is undetermined, the display control unit 33 outputs information indicating that the classification is undetermined, together with information regarding the classification scores. Thus, the display control unit 33 can suitably visualize the current state relating to the classification process of the lesion part.

In some embodiments, if the classification is undetermined, the display control unit 33 may instruct the audio output unit 16 to output a voice guidance or a predetermined warning sound informing the user that the classification is undetermined, instead of or in addition to the display control shown in the first display example. Thereby, the display control unit 33 can suitably allow the examiner to grasp that the classification is undetermined.

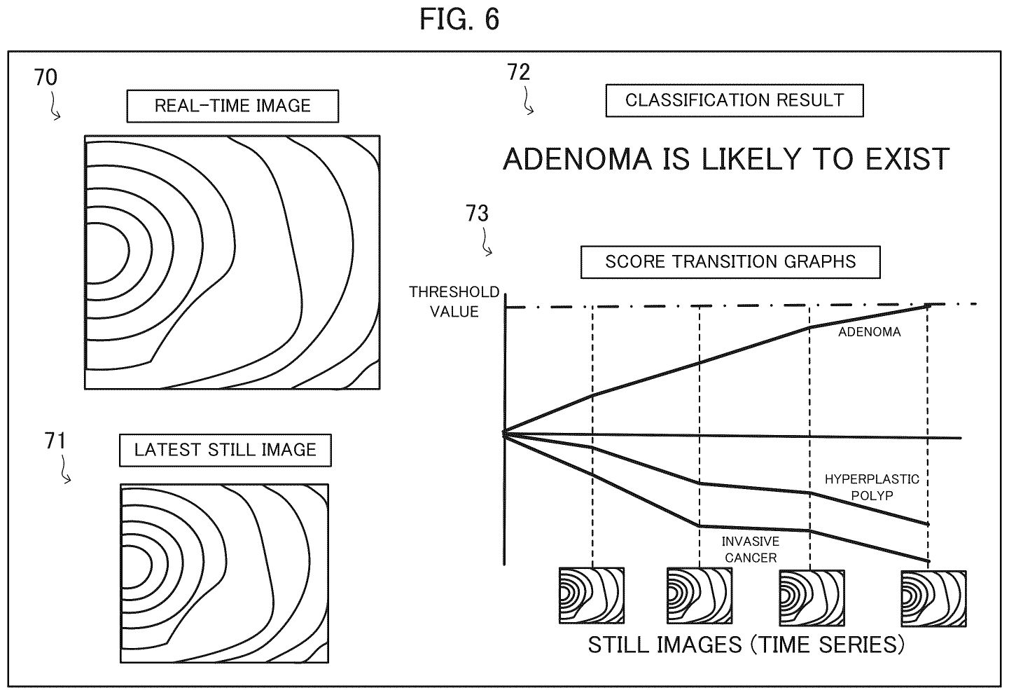

FIG. 6 shows a second display example of a display screen image displayed on the display device 2 in the endoscopic examination. In the second display example, the classification unit 32 determines that the classification score of the candidate class “adenoma” supplied from the score calculation unit 31 has reached the threshold value TH, and supplies the classification result indicating that it is classified into the candidate class “adenoma” to the display control unit 33. Then, in this case, in the classification result display area 72, based on the classification result described above, the display control unit 33 displays a text message indicating that an adenoma is likely to exist.

Thus, according to the second display example, once the classification is determined, the display control unit 33 outputs information indicating the classification result (text message in the classification result display area 72 in this case). Thus, the display control unit 33 can suitably notify the examiner of the classification result of the lesion part. In some embodiments, the display control unit 33 may instruct the audio output unit 16 to output the audio guidance or a predetermined warning sound to inform the examiner of the classification result. Thereby, the display control unit 33 also allows the examiner to grasp the classification result.

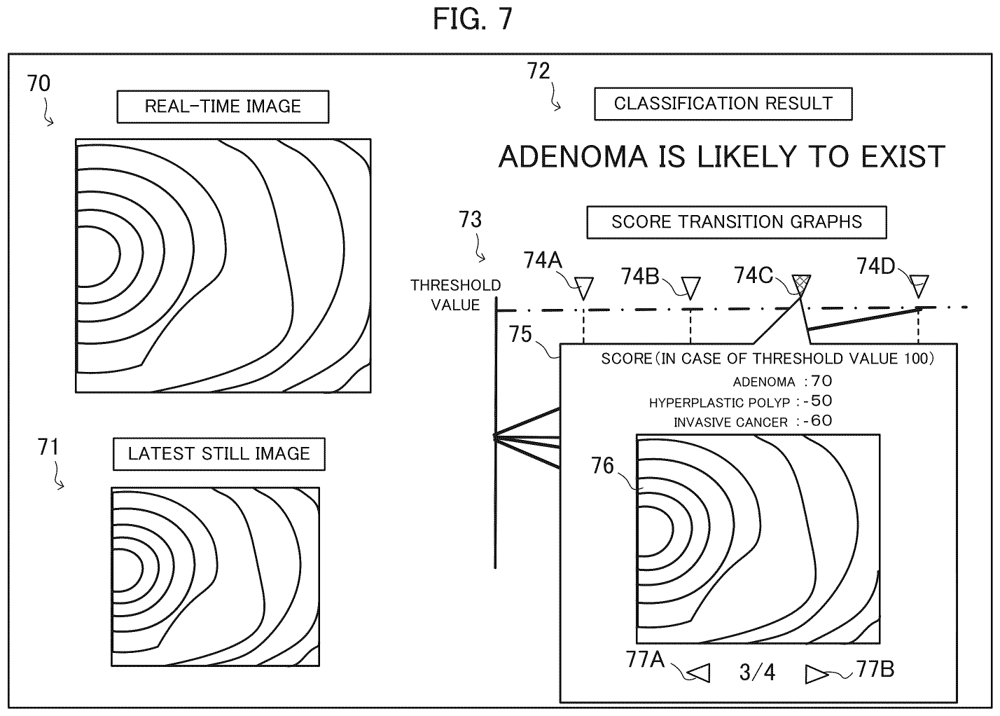

FIG. 7 shows a third display example of a display screen image displayed on the display device 2 in the endoscopic examination. In the third display example, the display control unit 33 enlarges and displays, in the score transition display area 73, the still image (i.e., lesion image) and the the classification score obtained at the time specified by the examiner.

Specifically, in the score transition display area 73, the display control unit 33 displays objects 74 (74A to 74D), which are associated with the respective acquisition times of the still images, in a selectable manner. Then, upon detecting that any one of the objects 74 has been selected, the display control unit 33 displays a still image (that is, a lesion image) and classification scores corresponding to the selected object 74 on the balloon window 75. In this case, the display control unit 33 detects that the object 74C has been selected and displays the still images 76 at the time corresponding to the object 74C and the classification scores of the respective candidate classes on the blowout window 75. This allows for presenting to the examiner a still image and classification scores at any point specified by the examiner.

Further, below the still image 76 displayed on the balloon window 75, the display control unit 33 displays the numerical value (3/4) representing the sequential order (third in this case) of the acquisition of the still image 76 among all the still images (four in this case) together with the switching buttons 77A and 77B. Here, the switching button 77A is an instruction button to display the preceding still image on the balloon window 75, and the switching button 77B is an instruction button to display the subsequent still image on the bubble window 75. Even by displaying such a user interface, the display control unit 33 can present the examiner with the still image and classification scores at any point in time specified by the examiner.

### (6) Processing Flow

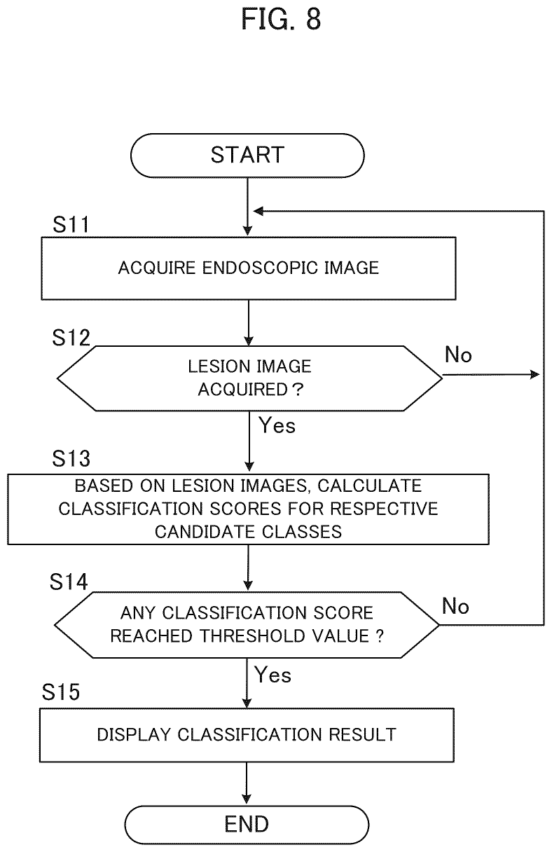

FIG. 8 is an example of a flowchart that is executed by the image processing device 1. The image processing device 1 repeatedly executes processing of the flowchart until the end of the endoscopic examination. For example, upon detecting a predetermined input or the like to the input unit 14 or the operation unit 36, the image processing device 1 determines that the endoscopic examination has been completed.

First, the lesion image acquisition unit 30 of the image processing device 1 acquires an endoscopic image Ia (step S11). In this instance, the lesion image acquisition unit 30 of the image processing device 1 receives the endoscopic image Ia from the endoscope 3 through the interface 13. The display control unit 33 executes a process of displaying the endoscopic image Ia acquired at step S11 on the display device 2.

Next, the lesion image acquisition unit 30 of the image processing device 1 determines whether or not a lesion image has been acquired (step S12). In this instance, the lesion image acquisition unit 30 acquires, as a lesion image, an endoscopic image Ia having a lesion part detected by the lesion detection model, or an endoscopic image Ia designated by the examiner using the operation unit 36 or the like. Then, if a lesion image has not been acquired (step S12; No), the lesion image acquisition unit 30 gets back to the process at step S11.

On the other hand, once the lesion image acquisition unit 30 determines that the lesion image has been acquired (step S12; Yes), the score calculation unit 31 of the image processing device 1 calculates the classification scores of the respective candidate classes at the current processing time on the basis of the lesion images obtained at step S12 at the current processing time and at the past processing times (step S13).

In calculating the classification scores at step S13, first, the score calculation unit 31 acquires, as the first calculation information, acquired lesion images acquired in the past according to the flowchart or their features, and calculates the likelihood ratio based on the first calculation information and the lesion image at the current process time acquired at step S12. The score calculation unit 31 stores the calculated likelihood ratio, and the lesion image acquired at step S12 or the features thereof as the first calculation information in the first calculation information storage unit D1. Then, the score calculation unit 31 calculates the integrated likelihood ratio based on the equation (1) by referring to the likelihood ratio stored in the first calculation information storage unit D1, and sets, as the classification score, the calculated integrated likelihood ratio or a value of a function having the integrated likelihood ratio as a variable. The score calculation unit 31 stores the calculated classification scores as the second calculation information in the second calculation information storage unit D2. The display control unit 33 may perform a process of displaying information on the classification scores calculated by the score calculation unit 31 on the display device 2.

Next, the classification unit 32 of the image processing device 1 determines whether or not the classification score of a candidate class has reached the threshold value TH (step S14). Then, upon determining that the classification score of a candidate class has reached the threshold value TH (step S14; Yes), the classification unit 32 determines the classification of the lesion part. Then, the display control unit 33 causes the display device 2 to display information based on the classification result generated by the classification unit 32 (step S15). On the other hand, upon determining that the classification score of any candidate class has not reached the threshold value TH (step S14; No), the classification unit 32 proceeds back to the process at step S11.

### (7) Modifications

Next, modifications suitable for the above-described example embodiment will be described. The following modifications may be applied to the example embodiment described above in any combination.

### First Modification

The image processing device 1 may simultaneously perform detection of the lesion part in the endoscopic image Ia and classification of the lesion part based on the classification scores calculated by the score calculation unit 31.

In this instance, the lesion image acquisition unit 30 supplies the endoscopic image Ia supplied from the endoscope 3 through the interface 13 to the score calculation unit 31 without selecting the lesion image. Then, the score calculation unit 31 calculates the classification scores of respective candidate classes. In this case, the score calculation unit 31 provides, as one of the candidate classes, a class (also referred to as “lesion non-detected class”) representing that there is no lesion part, and calculates the classification score for the lesion non-detected class in the same manner as the classification scores of the other candidate classes. Then, once the classification score of the lesion non-detected class has reached the threshold value TH, the classification unit 32 generates a classification result indicating that there is no lesion part in the endoscopic image Ia used for calculation of the classification scores.

Thus, according to the present modification, the image processing device 1 can suitably generate a classification result indicating the presence or absence of a lesion part without selecting a lesion image.

### (Second Modification)

The image processing device 1 may process, after the examination, a video configured by endoscopic images Ia generated during the endoscopic examination.

For example, if the video to be processed is designated based on user input by the input unit 14 at any timing after the examination, the image processing device 1 repeatedly performs processing of the flowchart shown in FIG. 8 for the time-series endoscopic images Ia constituting the video until it is determined that the video has ended.

### Second Example Embodiment

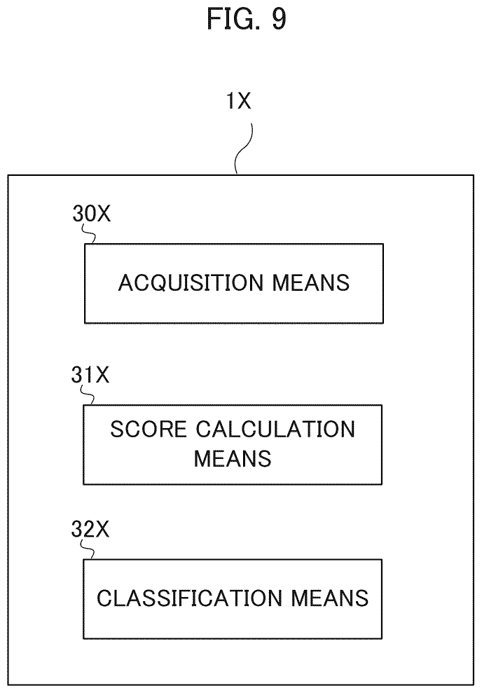

FIG. 9 is a block diagram of the image processing device 1X according to the second example embodiment. The image processing device 1X includes an acquisition means 30X, a score calculation means 31X, and a classification means 32X. The image processing device 1X may be configured by a plurality of devices.

The acquisition means 30X is configured to acquire an endoscopic image in which an examination target is photographed by a photographing unit provided in an endoscope. In this instance, the acquisition means 30X may immediately acquire the endoscopic image generated by the photographing unit, or may acquire, at a predetermined timing, the endoscopic image previously generated by the photographing unit and stored in a storage device. The endoscopic image acquired by the acquisition means 30X may be an endoscopic image (i.e., a lesion image in the first example embodiment) in which the lesion part appears. Examples of the acquisition means 30X include the lesion image acquisition unit 30 in the first example embodiment (including modifications, hereinafter the same).

The score calculation means 31X is configured to calculate scores on likelihoods of respective candidate classes corresponding to types of lesion, wherein the candidate classes are candidates for classification of an image group of the acquired endoscopic image. The “image group” is configured by one or more endoscopic images acquired by the acquisition means 30X. Examples of the score calculation means 31X include the score calculation unit 31 in the first example embodiment.

The classification means 32X is configured to perform the classification of the image group upon determining that at least one of the scores has reached a threshold value. Examples of the classification means 32X include the classification unit 32 in the first example embodiment.

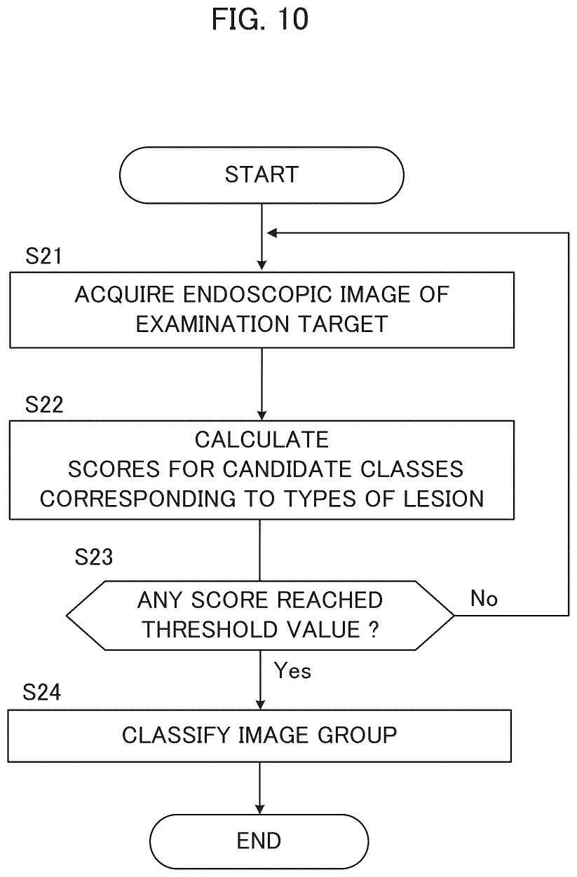

FIG. 10 is an example of a flowchart showing a processing procedure in the second example embodiment. First, the acquisition means 30X acquires an endoscopic image in which an examination target is photographed by a photographing unit provided in an endoscope (step S21). The score calculation means 31X calculates scores on likelihoods of respective candidate classes corresponding to types of lesion, wherein the candidate classes are candidates for classification of an image group of the acquired endoscopic image (step S22). Then, upon determining that at least one of the scores has reached a threshold value (step S23; Yes), the classification means 32X performs the classification of the image group (step S24). On the other hand, if none of the scores has reached the threshold value (step S23; No), the classification means 32X proceeds back to the process at step S21. In some embodiments, if none of the scores has reached the threshold value, the classification means 32X may additionally perform the process described in the first example embodiment. For example, once the image group includes a predetermined number or more of images, the classification means 32X may determine the classification to select the candidate class of the score closest to the threshold value or may restart the process of the flowchart with initialization of the image group.

According to the second example embodiment, the image processing device 1X is capable of accurately classifying the lesion part present in endoscopic images.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An image processing device comprising:
an acquisition means configured to acquire an endoscopic image in which an examination target is photographed by a photographing unit provided in an endoscope;
a score calculation means configured to calculate scores on likelihoods of respective candidate classes corresponding to types of lesion, wherein the candidate classes are candidates for classification of an image group of the acquired endoscopic image; and
a classification means configured to perform the classification of the image group upon determining that at least one of the scores has reached a threshold value.

### [Supplementary Note 2]

The image processing device according to Supplementary Note 1,
wherein, upon determining that none of the scores has reached the threshold value, the score calculation means is configured to update the scores based on the image group to which one or more endoscopic images acquired after the determination, and
wherein the classification means is configured to perform the classification of the image group upon determining that at least one of the updated scores has reached the threshold value.

### [Supplementary Note 3]

The image processing device according to Supplementary Note 1 or 2,
wherein once the number of the endoscopic images included in the image group has reached a predetermined number under a condition that it is determined that none of the scores has reached the threshold value, the classification means is configured to output a result of the classification indicating the candidate class which corresponds to the score closest to the threshold value.

### [Supplementary Note 4]

The image processing device according to Supplementary Note 1 or 2,
wherein once the number of the endoscopic images included in the image group has reached a predetermined number under a condition that it is determined that none of the scores has reached the threshold value, the classification means is configured to calculate the scores based on the image group of the endoscopic images which are acquired after the number of the endoscopic images included in the image group has reached the predetermined number.

### [Supplementary Note 5]

The image processing device according to Supplementary Note 1,
wherein the acquisition means is configured to acquire, among the endoscopic images outputted by the photographing unit, one or more lesion images that are the endoscopic images in which a lesion part suspected of the lesion appears, and
wherein the score calculation means is configured to calculate the scores based on the image group of the lesion images.

### [Supplementary Note 6]

The image processing device according to Supplementary Note 1, further comprising
an output control means configured to output information regarding the scores and a result of the classification by a display device or an audio output device.

### [Supplementary Note 7]

The image processing device according to Supplementary Note 6,
wherein the output control means is configured to display a diagram showing a transition of the scores for the respective candidate classes by the display device.

### [Supplementary Note 8]

The image processing device according to Supplementary Note 7,
wherein the output control means is configured to display, on the display device, the endoscopic images in time series included in the image group in association with the diagram.

[Supplementary Note 9]

The image processing device according to Supplementary Note 7,
wherein the output control means is configured to display, on the display device, the endoscopic image and the score corresponding to the time specified in the diagram.

### [Supplementary Note 10]

An image processing method executed by a computer, the image processing method comprising:
acquiring an endoscopic image in which an examination target is photographed by a photographing unit provided in an endoscope;
calculating scores on likelihoods of respective candidate classes corresponding to types of lesion, wherein the candidate classes are candidates for classification of an image group of the acquired endoscopic image; and
performing the classification of the image group upon determining that at least one of the scores has reached a threshold value.

### [Supplementary Note 11]

A storage medium storing a program executed by a computer, the program causing the computer to:
acquire an endoscopic image in which an examination target is photographed by a photographing unit provided in an endoscope;
calculate scores on likelihoods of respective candidate classes corresponding to types of lesion, wherein the candidate classes are candidates for classification of an image group of the acquired endoscopic image; and
perform the classification of the image group upon determining that at least one of the scores has reached a threshold value.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1X Image Processing Device
2 Display device
3 Endoscope
4 Server device
11 Processor
12 Memory
13 Interface
14 Input unit
15 Light source unit
16 Audio output unit
100 Endoscopic examination system

What is claimed is:

1. An image processing device comprising:
at least one memory configured to store instructions; and
at least one processor configured to execute the instructions to:
acquire an endoscopic image in which an examination target is photographed by an endoscope;
calculate scores on likelihoods of respective candidate classes corresponding to types of lesion, wherein the candidate classes are candidates for classification of an image group of the acquired endoscopic image; and
perform the classification of the image group upon determining that at least one of the scores has reached a threshold value.

2. The image processing device according to claim 1,
wherein, upon determining that none of the scores has reached the threshold value, the at least one processor is configured to execute the instructions to update the scores based on the image group to which one or more endoscopic images acquired after the determination, and
wherein the at least one processor is configured to execute the instructions to perform the classification of the image group upon determining that at least one of the updated scores has reached the threshold value.

3. The image processing device according to claim 1,
wherein once the number of the endoscopic images included in the image group has reached a predetermined number under a condition that it is determined that none of the scores has reached the threshold value, the at least one processor is configured to execute the instructions to output a result of the classification indicating the candidate class which corresponds to the score closest to the threshold value.

4. The image processing device according to claim 1,
wherein once the number of the endoscopic images included in the image group has reached a predetermined number under a condition that it is determined that none of the scores has reached the threshold value, the at least one processor is configured to execute the instructions to calculate the scores based on the image group of the endoscopic images which are acquired after the number of the endoscopic images included in the image group has reached the predetermined number.

5. The image processing device according to claim 1,
wherein the at least one processor is configured to execute the instructions to acquire, among the endoscopic images outputted by the endoscope, one or more lesion images that are the endoscopic images in which a lesion part suspected of the lesion appears, and
wherein the at least one processor is configured to execute the instructions to calculate the scores based on the image group of the lesion images.

6. The image processing device according to claim 1,
wherein the at least one processor is configured to execute the instructions to output information regarding the scores and a result of the classification by a display device or an audio output device.

7. The image processing device according to claim 6,
wherein the at least one processor is configured to execute the instructions to display a diagram showing a transition of the scores for the respective candidate classes by the display device.

8. The image processing device according to claim 7,
wherein the at least one processor is configured to execute the instructions to display, on the display device, the endoscopic images in time series included in the image group in association with the diagram.

9. The image processing device according to claim 7,
wherein the at least one processor is configured to execute the instructions to display, on the display device, the endoscopic image and the score corresponding to the time specified in the diagram.

10. An image processing method executed by a computer, the image processing method comprising:
acquiring an endoscopic image in which an examination target is photographed by an endoscope;
calculating scores on likelihoods of respective candidate classes corresponding to types of lesion, wherein the candidate classes are candidates for classification of an image group of the acquired endoscopic image; and
performing the classification of the image group upon determining that at least one of the scores has reached a threshold value.

11. A non-transitory computer readable storage medium storing a program executed by a computer, the program causing the computer to:
acquire an endoscopic image in which an examination target is photographed by an endoscope;
calculate scores on likelihoods of respective candidate classes corresponding to types of lesion, wherein the candidate classes are candidates for classification of an image group of the acquired endoscopic image; and
perform the classification of the image group upon determining that at least one of the scores has reached a threshold value.
